(19) United States
(12) Patent Application Publication
(10) Pub. No.: US 2007/0151569 A1
(43) Pub. Date: Jul. 5, 2007

(54) METHODS FOR ACHIEVING AND MAINTAINING WEIGHT LOSS

(76) Inventors: Steven J. Catani, Athens, GA (US); Steven D. Clarke, North Brunswick, NJ (US); Thomas E. Sox, Ambler, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/539,282

(22) Filed: Oct. 6, 2006

Related U.S. Application Data
(63) Continuation-in-part of application No. 11/245,762, filed on Oct. 7, 2005.

(51) Int. Cl. A61K 47/00; A61B 19/00

(52) U.S. Cl. 128/898; 424/439

(57) ABSTRACT

Methods for achieving weight loss goals and maintaining weight loss including selecting an appropriate weight loss program that provides a caloric deficit of not more than 350 kilocalories per day, and concurrently ingesting a composition comprising effective amounts of a combination of an anionic soluble fiber and a multivalent cation source during the weight loss program. Also, a method for producing weight loss including identifying an individual in need, providing the individual a weight loss program that provides a caloric deficit of not more than 350 kilocalories per day, and directing the individual to ingest a composition comprising an effective amount of a combination of anionic soluble fiber and a multivalent cation.

### CROSS-REFERENCE TO RELATED APPLICATION

This application is a continuation-in-part application of copending U.S. patent application Ser. No. 11/245,762, filed Oct. 7, 2005.

### FIELD OF THE INVENTION

The present invention is directed to a method for achieving weight loss and maintaining weight loss.

### BACKGROUND OF THE INVENTION

Diabetes and obesity are common ailments in the United States and other Western cultures. A study by researchers at RTI International and the Centers for Disease Control estimated that U.S. obesity-attributable medical expenditures reached $75 billion in 2003. Obesity has been shown to promote many chronic diseases, including type 2 diabetes, cardiovascular disease, several types of cancer, and gallbladder disease.

Adequate dietary intake of soluble fiber has been associated with a number of health benefits, including decreased blood cholesterol levels, improved glycemic control, and the induction of satiety and satiation in individuals. Consumers have been resistant to increasing soluble fiber amounts in their diet, however, often due to the negative organoleptic characteristics, such as, sliminess, excessive viscosity, excessive dryness and poor flavor, that are associated with food products that include soluble fiber.

What is needed are weight loss methods and weight maintenance using, among other things, ingestible compositions having fibers and cations.

### SUMMARY OF THE INVENTION

The present invention solves those needs. One embodiment of the present invention is directed to a method for achieving weight loss goals and maintaining weight loss comprising, consisting of, and/or consisting essentially of the steps of first, selecting an appropriate weight loss program and identifying a weight loss goal; followed by, second, participating in the weight loss program until the weight loss goal is achieved; third, ending the weight loss program participation; and fourth, consuming an ingestible composition at regular intervals beginning from about 1 to about 48 hours after ending the weight loss program, the ingestible composition comprising an effective amount of a multivalent cation and an effective amount of an soluble anionic fiber.

Another embodiment of the present invention is directed to a method for achieving and maintaining weight loss comprising, consisting of, and/or consisting essentially of the steps of first, selecting an appropriate weight loss program and identifying a weight loss goal of at least 5% of total body weight, followed by participating in the weight loss program until the weight loss goal is achieved; ending the weight loss program participation; and consuming an ingestible composition comprising a solid component and a fluid component at regular intervals between breakfast and lunch, lunch and dinner, or both, beginning from about 1 to about 48 hours after ending the weight loss program, the ingestible composition comprising, consisting of, and/or consisting essentially of an effective amount of a calcium source in the fluid component and from bout 0.5 g to about 10 g total soluble anionic fiber per serving wherein the soluble anionic fiber is a mixture of alginate and pectin in the solid component.

Yet another embodiment of the present invention is directed to a method for achieving and maintaining weight comprising, consisting of, and/or consisting essentially of the steps of engaging in a weight loss program that involves low intensity counseling, reducing daily caloric intake below maintenance levels by less than 500 kilocalories per day, and consuming during the weight loss program an ingestible composition comprising, consisting of, and/or consisting essentially of a weight loss promoting effective amount of a combination of soluble anionic fiber and a divalent cation source.

### BRIEF DESCRIPTION OF DRAWINGS

FIG. 1 is a graph depicting the effects of an embodiment of the present invention on intestinal viscosity.

### DETAILED DESCRIPTION OF THE INVENTION

As used herein, unless indicated otherwise, the terms “alginate,” “pectin,” “carrageenan,” “polygeenan,” or “gellan” refers to all forms (e.g., protonated or salt forms, such as sodium, potassium, and ammonium salt forms and having varying average molecular weight ranges) of the soluble anionic fiber type.

As used herein, unless indicated otherwise, the term “alginic acid” includes not only the material in protonated form but also the related salts of alginate, including but not limited to sodium, potassium, and ammonium alginate.

As used herein, unless indicated otherwise, the term “protected” means that the source has been treated in such a way, as illustrated below, to delay (e.g., until during or after ingestion or until a certain pH range has been reached) reaction of the at least one multivalent cation with the soluble anionic fiber as compared to an unprotected multivalent cation.

As used herein, unless indicated otherwise, the term SE or Satiety Efficiency Index means, unless otherwise defined, caloric reduction in a given meal due to preload divided by the caloric value of the preload. For example, if a person consumes a 1000 calorie lunch without ingesting a preload, but consumes a 900 calorie lunch after ingesting a 200 calorie preload, the preload would have a 0.50 or 50% SE. Another example is a person consumes a 1000 calorie lunch without ingesting a preload, but consumes a 800 calorie lunch after ingesting a 100 calorie preload, the preload would have a 2.0 or 200% SE. As can be seen, the greater the SE, the greater the effect of the preload on the next meal.

As used herein, unless indicated otherwise, the term “low intensity counseling” means counseling, in the context of a weight loss or weight maintenance program, that comprises maintaining, under the auspices of a counselor or health care professional, food intake or caloric intake diaries, recording physical activity in the same or separate diaries, recording the steps taken with a pedometer or a similar device, reviewing these data on a periodic basis with a health care professional or a weight loss counselor, and being weighed on a periodic basis by such professional or counselor to ascertain progress. In the context of low intensity counseling, the periodic review of data and weighing may occur at intervals of about once per week to about once per quarter, more preferably every two to six week, and most preferably monthly.

As used herein, unless indicated otherwise, the term “caloric deficit” is, in a person undergoing weight loss, the difference in the number of calories of energy actually consumed, versus the number of calories that would have to be consumed to maintain a stable weight. For example, an individual might require 2000 kcal of energy intake per day to maintain stable weight at a fixed level of physical activity. During a weight loss program, this same individual might consume 1700 kcal per day, while maintaining the same fixed level of physical activity. In this example, the daily caloric deficit would be 300 kcal. A caloric deficit can also be achieved by maintaining a constant daily energy intake (as measured by kcal of energy ingested) but increasing caloric expenditure through physical activity. Caloric deficit may be determined by measuring the mass units of weight loss of an individual over an interval of time, multiplying the mass of weight loss by the caloric content of a unit of body mass in an overweight or obese individual, and dividing that product by the units of time in the interval. For example, assume that an individual lost two kg over an eight-week interval. Assuming that each kilogram of lost weight was equivalent to 8000 kcal, then the caloric deficit was 16,000 kcal over 56 days, or the caloric deficit of 286 kcal per day.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

As used herein, a recitation of a range of values is merely intended to serve as a shorthand method of referring individually to each separate value falling within the range, and each separate value is incorporated into the specification as if it were individually recited herein.

The inventors were surprised to discover that the compositions of this invention reduce food intake at consumption levels of dietary fiber much lower than the levels that have previously been reported to reduce food intake. The inventors believe that this arises from the enhanced viscosity produced by the interactions of soluble multivalent cation and at least one soluble anionic fiber.

Soluble Anionic Fiber

Any soluble anionic fiber should be acceptable for the purposes of this invention. Suitable soluble anionic fibers include alginate, pectin, gellan, soluble fibers that contain carboxylate substituents, carrageenan, polygeenan, and marine algae-derived polymers that contain sulfate substituents.

Also included within the scope of soluble anionic fibers are other plant derived and synthetic or semisynthetic polymers that contain sufficient carboxylate, sulfate, or other anionic moieties to undergo gelling in the presence of sufficient levels of multivalent cation.

At least one source of soluble anionic fiber may be used in these compositions, and the at least one source of soluble anionic fiber may be combined with at least one source of soluble fiber that is uncharged at neutral pH. Thus, in certain cases, two or more soluble anionic fibers types are included, such as, alginate and pectin, alginate and gellan, or pectin and gellan. In other cases, only one type of soluble anionic fiber is used, such as only alginate, only pectin, only carrageenan, or only gellan.

Soluble anionic fibers are commercially available, e.g., from ISP (Wayne, N.J.), TIC Gums, and CP Kelco.

An alginate can be a high guluronic acid alginate. For example, in certain cases, an alginate can exhibit a higher than 1:1 ratio of guluronic to mannuronic acids, such as in the range from about 1.2:1 to about 1.8:1, e.g., about 1.3:1, about 1.4:1, about 1.5:1, about 1.6:1, or about 1.7:1 or any value therebetween. Examples of high guluronic alginates (e.g., having a higher than 1:1 g:m ratios) include Manugel LBA, Manugel GHB, and Manugel DBP, which each have a g:m ratio of about 1.5.

While not being bound by theory, it is believed that high guluronic alginates can cross-link through multivalent cations, e.g., calcium ions, to form gels at the low pH regimes in the stomach. High guluronic alginates are also believed to electrostatically associate with pectins and/or gellans at low pHs, leading to gellation. In such cases, it may be useful to delay the introduction of multivalent cations until after formation of the mixed alginate/pectin or alginate/gellan gel, as multivalent cationic cross-links may stabilize the mixed gel after formation.

In other cases, an alginate can exhibit a ratio of guluronic to mannuronic acids (g:m ratio) of less than about 1:1, e.g., about 0.8:1 to about 0.4:1, such as about 0.5:1, about 0.6:1, or about 0.7:1 or any value therebetween. Keltone LV and Keltone HV are examples of high-mannuronic acids (e.g., having a g:m ratio of less than 1:1) having g:m ratios ranging from about 0.6:1 to about 0.7:1.

Methods for measuring the ratio of guluronic acids to mannuronic acids are known by those having ordinary skill in the art.

An alginate can exhibit any number average molecular weight range, such as a high molecular weight range (about 2.05×10⁵ to about 3×10⁵ Daltons or any value therebetween; examples include Manugel DPB, Keltone HV, and TIC 900 Alginate); a medium molecular weight range (about 1.38×10⁵ to about 2×10⁵ Daltons or any value therebetween; examples include Manugel GHB); or a low molecular weight range (about 2×10⁴ to about 1.35×10⁵ Daltons or any value therebetween; examples include Manugel LBA and Manugel LBB). Number average molecular weights can be determined by those having ordinary skill in the art, e.g., using size exclusion chromatography (SEC) combined with refractive index (RI) and multi-angle laser light scattering (MALLS).

In certain embodiments of a formed food product, a low molecular weight alginate can be used (e.g., Manugel LBA), while in other cases a mixture of low molecular weight (e.g., Manugel LBA) and high molecular weight (e.g., Manugel DPB, Keltone HV) alginates can be used. In other cases, a mixture of low molecular weight (e.g., Manugel LBA) and medium molecular weight (e.g., Manugel GHB) alginates can be used. In yet other cases, one or more high molecular weight alginates can be used (e.g., Keltone HV, Manugel DPB).

A pectin can be a high-methoxy pectin (e.g., having greater than 50% esterified carboxylates), such as ISP HM70LV and CP Kelco USPL200. A pectin can exhibit any number average molecular weight range, including a low molecular weight range (about 1×10⁵ to about 1.20×10⁵ Daltons, e.g., CP Kelco USPL200), medium molecular weight range (about 1.25×10⁵ to about 1.45×10⁵, e.g., ISP HM70LV), or high molecular weight range (about 1.50×10⁵ to about 1.80×10⁵, e.g., TIC HM Pectin). In certain cases, a high-methoxy pectin can be obtained from pulp, e.g., as a by-product of orange juice processing.

A gellan soluble anionic fiber can also be used. Gellan fibers form strong gels at lower concentrations than alginates and/or pectins, and can cross-link with multivalent cation cations. For example, gellan can form gels with sodium, potassium, magnesium, and calcium. Gellans for use in the invention include Kelcogel, available commercially from CP Kelco.

Fiber blends as described herein can also be used in the preparation of a solid ingestible composition like a formed food product where the fiber blend is a source of the soluble anionic fiber. A useful fiber blend can include an alginate soluble anionic fiber and a pectin soluble anionic fiber. A ratio of total alginate to total pectin in a blend can be from about 8:1 to about 5:1, or any value therebetween, such as about 7:1, about 6.5:1, about 6.2:1, or about 6.15:1. A ratio of a medium molecular weight alginate to a low molecular weight alginate can range from about 0.65:1 to about 2:1, or any value therebetween.

An alginate soluble anionic fiber in a blend can be a mixture of two or more alginate forms, e.g., a medium and low molecular weight alginate. In certain cases, a ratio of a medium molecular weight alginate to a low molecular weight alginate is about 0.8:1 to about 0.9:1. The fiber blend combining low and medium molecular weight alginates with high methoxy pectin can be from about 0 to about 3 grams. The preferred range for both would be about 1 to about 2 grams.

The at least one soluble anionic fiber may be treated before, during, or after incorporation into an ingestible composition. For example, the at least one soluble anionic fiber can be processed, e.g., extruded, roll-dried, freeze-dried, dry blended, roll-blended, agglomerated, coated, or spray-dried.

For solid forms, a variety of formed shapes of food products can be prepared by methods known to those having ordinary skill in the art, extruding, molding, pressing, wire-cutting. For example, a single or double screw extruder can be used. Typically, a feeder meters in the raw ingredients to a barrel that includes the screw(s). The screw(s) conveys the raw material through the die that shapes the final product. Extrusion can take place under high temperatures and pressures or can be a non-cooking, forming process. Extruders are commercially available, e.g., from Buhler, Germany. Extrusion can be cold or hot extrusion.

Other processing methods are known to those having skilled in the art.

The amount of the at least one soluble anionic fiber included can vary, and will depend on the type of ingestible composition and the type of soluble anionic fiber used. For example, typically a solid ingestible composition will include from about 0.5 g to about 10 g total soluble anionic fiber per serving or any value therebetween. A preferred range of fiber intake in the compositions of this invention is about 0.25 g to about 5 g per serving, more preferably about 0.5 to about 3 g per serving, and most preferably about 1.0 to about 2.0 g per serving. In certain cases, a formed food product can include a soluble anionic fiber at a total amount from about 22% to about 40% by weight of the formed product or any value therebetween. In other cases, a formed food product can include an soluble anionic fiber in a total amount of from about 4% to about 15% or any value therebetween, such as when only gellan is used. In yet other cases, a formed food product can include a soluble anionic fiber at a total amount of from about 18% to about 25% by weight, for example, when combinations of gellan and alginate or gellan and pectin are used.

In addition to the at least one soluble anionic fiber, a solid ingestible composition can include ingredients that may be treated in a similar manner as the at least one soluble anionic fiber. For example, such ingredient can be co-extruded with the soluble anionic fiber, co-processed with the soluble anionic fiber, or co-spray-dried with the soluble anionic fiber. Such treatment can help to reduce sliminess of the ingestible composition in the mouth and to aid in hydration and gellation of the fibers in the stomach and/or small intestine. Without being bound by any theory, it is believed that co-treatment of the soluble anionic fiber(s) with such ingredient prevents early gellation and hydration of the fibers in the mouth, leading to sliminess and unpalatability. In addition, co-treatment may delay hydration and subsequent gellation of the soluble anionic fibers (either with other soluble anionic fibers or with multivalent cations) until the ingestible composition reaches the stomach and/or small intestine, providing for the induction of satiety and/or satiation.

Additional ingredients can be hydrophilic in nature, such as starch, protein, maltodextrin, and inulin. Other additional ingredients can be insoluble in water (e.g., cocoa solids, corn fiber) and/or fat soluble (vegetable oil), or can be flavor modifiers such as sucralose. For example, a formed food product can include from about 5 to about 80% of a cereal ingredient, such as about 40% to about 68% of a cereal ingredient. A cereal ingredient can be rice, corn, wheat, sorghum, oat, or barley grains, flours, or meals. Thus, a formed food product can include about 40% to about 50%, about 50% to about 58%, about 52% to about 57%, or about 52%, about 53%, about 54%, about 55%, about 56%, or about 56.5% of a cereal ingredient. In one embodiment, about 56.5% of rice flour is included.

An ingestible composition can also include a protein source. A protein source can be included in the composition or in a formed food product. For example, a formed food product can include a protein source at about 2% to about 20% by weight, such as about 3% to about 8%, about 3% to about 5%, about 4% to about 7%, about 4% to about 6%, about 5% to about 7%, about 5% to about 15%, about 10% to about 18%, about 15% to about 20%, or about 8% to about 18% by weight. A protein can be any known to those having ordinary skill in the art, e.g., rice, milk, egg, wheat, whey, soy, gluten, or soy flour. In some cases, a protein source can be a concentrate or isolate form.

Multivalent Cation

The compositions and associated methods of this invention include a source of at least one multivalent cation in an amount sufficient to cause an increase in viscosity of the soluble anionic fiber. A source of at least one multivalent cation may be incorporated into an ingestible composition provided herein, or can consumed as a separate food article either before, after, or simultaneously with an ingestible composition.

Any multivalent cation may be used in the present invention, e.g., multivalent, trivalent, and the like. Multivalent cations useful in this invention include, calcium, magnesium, aluminum, manganese, iron, nickel, copper, zinc, strontium, barium, bismuth, chromium, vanadium, lanthanum, their salts and mixtures thereof. Salts of the multivalent cations may be organic acid salts that include formate, fumarate, acetate, propionate, butyrate, caprylate, valerate, lactate, citrate, malate and gluconate. Also included are highly soluble inorganic salts such as chlorides or other halide salts.

In certain compositions, one or more particular multivalent cations may be used with certain soluble anionic fibers, depending on the composition and gel strength desired. For example, for ingestible alginate compositions, calcium may be used to promote gellation. For gellan compositions, one or more of calcium and magnesium may be used.

The at least one multivalent cation can be unable to, or be limited in its ability to, react with the at least one soluble anionic fiber in the ingestible composition until during or after ingestion. For example, physical separation of the at least one multivalent cation from the at least one soluble anionic fiber, e.g., as a separate food article or in a separate matrix of the ingestible composition from the at least one soluble anionic fiber, can be used to limit at least one multivalent cation's ability to react. In other cases, the at least one multivalent cation is limited in its ability to react with the at least one soluble anionic fiber by protecting the source of at least one multivalent cation until during or after ingestion. Thus, the at least one multivalent cation, such as, a protected multivalent cation, can be included in the ingestible composition or can be included as a separate food article composition, e.g., for separate ingestion either before, during, or after ingestion of an ingestible composition.

Typically, a separate food article containing the source of at least one multivalent cation would be consumed in an about four hour time window flanking the ingestion of an ingestible composition containing the at least one soluble anionic fiber. In certain cases, the window may be about three hours, or about two hours, or about one hour. In other cases, the separate food article may be consumed immediately before or immediately after ingestion of an ingestible composition, e.g., within about fifteen minutes, such as within about 10 minutes, about 5 minutes, or about 2 minutes. In other cases, a separate food article containing at least one multivalent cation can be ingested simultaneously with an ingestible composition containing the at least one soluble anionic fiber, e.g., a snack chip composition where some chips include at least one multivalent cation and some chips include the at least one soluble anionic fiber.

In one embodiment, at least one multivalent cation can be included in an ingestible composition in a different food matrix from a matrix containing a soluble anionic fiber. For example, a source of at least one multivalent cation, such as a calcium salt, can be included in a separate matrix of a solid ingestible composition from the matrix containing the at least one soluble anionic fiber. Thus, means for physical separation of an soluble anionic fiber (e.g., within a snack bar or other formed food product) from a source of at least one multivalent cation are also contemplated, such as by including the source of at least one multivalent cation in a matrix such as a frosting, water and fat based icing, coating, decorative topping, drizzle, chip, chunk, swirl, filling, or interior layer. In one embodiment, a source of at least one multivalent cation, such as a protected multivalent cation source, can be included in a snack bar matrix that also contains an extruded crispy matrix that contains the soluble anionic fiber. In such a case, the source of at least one multivalent cation is in a separate matrix than the crispy matrix containing the soluble anionic fiber. In another embodiment, a source of at least one multivalent cation can be included in a gel layer or phase, e.g., a jelly or jam.

One multivalent cation source is multivalent cation salts. Typically, a multivalent cation salt can be selected from the following salts: citrate, tartrate, malate, formate, lactate, gluconate, phosphate, carbonate, sulfate, chloride, acetate, propionate, butyrate, caprylate, valerate, fumarate, adipate, and succinate. In certain cases, a multivalent cation salt is a calcium salt. A calcium salt can have a solubility of >1% w/vol in water at pH 7 at 20° C. A calcium salt can be, without limitation, calcium citrate, calcium tartrate, calcium malate, calcium lactate, calcium gluconate, dicalcium phosphate dihydrate, anhydrous calcium diphosphate, dicalcium phosphate anhydrous, calcium carbonate, calcium sulfate dihydrate, calcium sulfate anhydrous, calcium chloride, calcium acetate monohydrate, monocalcium phosphate monohydrate, and monocalcium phosphate anhydrous.

The source of at least one multivalent cation can be a protected source.

A number of methods can be used to protect a source of at least one multivalent cation. For example, microparticles or nanoparticles having double or multiple emulsions, such as water/oil/water (“w/o/w”) or oil/water/oil (“o/w/o”) emulsions, of at least one multivalent cation and an soluble anionic fiber can be used. In one embodiment, a calcium alginate microparticle or nanoparticle is used. For example, a calcium chloride solution can be emulsified in oil, which emulsion can then be dispersed in a continuous water phase containing the anionic alginate soluble fiber. When the emulsion breaks in the stomach, the calcium can react with the alginate to form a gel.

A microparticle can have a size from about 1 to about 15 μM (e.g., about 5 to about 10 μM, or about 3 to about 8 μM). A nanoparticle can have a size of about 11 to about 85 nm (e.g., about 15 to about 50 nm, about 30 to about 80 nm, or about 50 to about 75 nm). The preparation of multiple or double emulsions, including the choice of surfactants and lipids, is known to those having ordinary skill in the art.

In another embodiment, nanoparticles of calcium alginate are formed by preparing nanodroplet w/o microemulsions of CaCl₂ in a solvent and nanodroplet w/o microemulsions of alginate in the same solvent. When the two microemulsions are mixed, nanoparticles of calcium alginate are formed. The particles can be collected and dispersed, e.g., in a fluid ingestible composition. As the particle size is small (<100 nm), the particles stay dispersed (e.g., by Brownian motion), or can be stabilized with a food grade surfactant. Upon ingestion, the particles aggregate and gel.

In other embodiments, a liposome containing a source of at least one multivalent cation can be included in an ingestible composition. For example, a calcium-containing liposome can be used. The preparation of liposomes containing multivalent cations is well known to those having ordinary skill in the art; see ACS Symposium Series, 1998 709:203-211; Chem. Mater. 1998 (109-116). Cochelates can also be used, e.g., as described in U.S. Pat. No. 6,592,894 and U.S. Pat. No. 6,153,217. The creation of cochelates using multivalent cations such as calcium can protect the multivalent cations from reacting with the soluble anionic fiber within the aqueous phase of an ingestible composition, e.g., by wrapping the multivalent cations in a hydrophobic lipid layer, thus delaying reaction with the fiber until digestion of the protective lipids in the stomach and/or small intestine via the action of lipases.

In certain cases, a multivalent cation-containing carbohydrate glass can be used, such as a calcium containing carbohydrate glass. A carbohydrate glass can be formed from any carbohydrate such as, without limitation, sucrose, trehalose, inulin, maltodextrin, corn syrup, fructose, dextrose, and other mono-, di-, or oligo-saccharides using methods known to those having ordinary skill in the art; see, e.g., WO 02/05667. A carbohydrate glass can be used, e.g., in a coating or within a food matrix.

Ingestible Compositions

Compositions of the present invention can be in any form, fluid or solid. Fluids can be beverages, including shake, liquado, and smoothie. Fluids can be from low to high viscosity.

Solid forms ca formed or not. Solid forms may include bread, cracker, bar, mini-bars, cookie, confectioneries, e.g., nougats, toffees, fudge, caramels, hard candy enrobed soft core, muffins, cookies, brownies, cereals, chips, snack foods, bagels, chews, crispies, and nougats, pudding, jelly, and jam. Solids can have densities from low to high.

Fluids

Fluid ingestible compositions can be useful for, among other things, aiding in weight loss programs, e.g., as meal replacement beverages or diet drinks. Fluid ingestible compositions can provide from about 0.5 g to about 10 g of soluble anionic fiber per serving, or any value therebetween. For example, in certain cases, about 1 g, 2 g, 3 g, 4 g, 5 g, 6 g, 7 g, 8 g, or 9 g of at least one soluble anionic fiber are provided per serving.

A fluid ingestible composition may include an alginate soluble anionic fiber and/or a pectin soluble anionic fiber. In certain cases, an alginate soluble anionic fiber and a pectin soluble anionic fiber are used. A fiber blend as described herein can be used to provide the alginate soluble anionic fiber and/or the pectin soluble anionic fiber. An alginate and pectin can be any type and in any form, as described previously. For example, an alginate can be a high, medium, or low molecular weight range alginate, and a pectin can be a high-methoxy pectin. Also as indicated previously, two or more alginate forms can be used, such as a high molecular weight and a low molecular weight alginate, or two high molecular weight alginates, or two low molecular weight alginates, or a low and a medium molecular weight alginate, etc. For example, Manugel GHB alginate and/or Manugel LBA alginate can be used. In other cases, Manugel DPB can be used. Genu Pectin, USPL200 (a high-methoxy pectin) can be used as a pectin. In certain cases, potassium salt forms of an soluble anionic fiber can be used, e.g., to reduce the sodium content of an ingestible composition.

A fluid ingestible composition includes alginate and/or pectin in a total amount of about 0.3% to about 5% by weight, or any value therebetween, e.g., about 1.25% to about 1.9%; about 1.4% to about 1.8%; about 1.0% to about 2.2%, about 2.0% to about 4.0%, about 3.0%, about 4.0%, about 2.0%, about 1.5%, or about 1.5% to about 1.7%. Such percentages of total alginate and pectin can yield about 2 g to about 8 g of fiber per 8 oz. serving, e.g., about 3 g, about 4 g, about 5 g, about 6 g, or about 7 g fiber per 8 oz. serving. In other cases, about 4 g to about 8 g of fiber (e.g., about 5 g, about 6 g, or about 7 g) per 12 oz. serving can be targeted. In some embodiments, about 1.7% fiber by weight of a fluid ingestible composition is targeted.

In some cases, a fluid ingestible composition includes only alginate as a soluble anionic fiber. In other cases, alginate and pectin are used. A ratio of alginate to pectin (e.g., total alginate to total pectin) in a fluid ingestible composition can range from about 8:1 to about 1:8, and any ratio therebetween (e.g., alginate:pectin can be in a ratio of about 1:1, about 1.2:1, about 1.3:1, about 1.4:1, about 1.5:1, about 1.6:1, about 1.62:1, about 1.7:1, about 1.8:1, about 1.9:1, about 2:1, about 3:1, about 4:1, about 5:1, about 5.3:1, about 5.6:1, about 5.7:1, about 5.8:1, about 5.9:1, about 6:1, about 6.1:1, about 6.5:1, about 7:1, about 7.5:1, about 7.8:1, about 2:3, about 1:4, or about 0.88:1). In cases where alginate and pectin are in a ratio of about 0.5:1 to about 2:1, it is believed that pectin and alginate electrostatically associate with one another to gel in the absence of multivalent cations; thus, while not being bound by theory, it may be useful to delay the introduction of multivalent cations until after such gel formation. In other cases, where the ratio of alginate to pectin is in the range from about 3:1 to about 8:1, it may be useful to include a multivalent cation source, such as, a calcium source (e.g., to crosslink the excess alginate) to aid gel formation in the stomach. In these cases, the inventors believe, while not being bound by any theory, that the lower amount of pectin protects the alginate from precipitating as alginate at the low pHs of the stomach environment, while the multivalent cation source cross-links and stabilizes the gels formed.

A fluid ingestible composition can have a pH from about 3.9 to about 4.5, e.g., about 4.0 to about 4.3 or about 4.1 to about 4.2. At these pHs, it is believed that the fluid ingestible compositions are above the pKas of the alginate and pectin acidic subunits, minimizing precipitation, separation, and viscosity of the solutions. In some cases, malic, phosphoric, and citric acids can be used to acidify the compositions. In some cases, a fluid ingestible composition can have a pH of from about 5 to about 7.5. Such fluid ingestible compositions can use pH buffers known to those having ordinary skill in the art.

Sweeteners for use in a fluid ingestible composition can vary according to the use of the composition. For beverages, low glycemic sweeteners may be preferred, including trehalose, isomaltulose, aspartame, saccharine, and sucralose. Sucralose can be used alone in certain formulations. The choice of sweetener will impact the overall caloric content of a fluid ingestible composition. In certain cases, fluid ingestible compositions can be targeted to have 40 calories/12 oz serving.

A fluid ingestible composition can demonstrate gel strengths of about 20 to about 250 grams force (e.g., about 60 to about 240, about 150 to about 240, about 20 to 30, about 20 to about 55, about 50 to 200; about 100 to 200; and about 175 to 240), as measured in a static gel strength assay. Gel strengths can be measured in the presence and absence of a multivalent cation source, such as, a calcium source.

A fluid ingestible composition can exhibit a viscosity in the range of from about 15 to about 100 cPs, or any value therebetween, at a shear rate of about 10⁻⁵, e.g., about 17 to about 24; about 20 to about 25; about 50 to 100, about 25 to 75, about 20 to 80, or about 15 to about 20 cPs. Viscosity can be measured by those skilled in the art, e.g., by measuring flow curves of solutions with increasing shear rate using a double gap concentric cylinder fixture (e.g., with a Parr Physica Rheometer).

A fluid ingestible composition can include a multivalent cation sequestrant, e.g., to prevent premature gellation of the soluble anionic fibers. A multivalent cation sequestrant can be selected from EDTA and its salts, EGTA and its salts, sodium citrate, sodium hexametaphosphate, sodium acid pyrophosphate, trisodium phosphate anhydrous, tetrasodium pyrophosphate, sodium tripolyphosphate, disodium phosphate, sodium carbonate, and potassium citrate. A multivalent cation sequestrant can be from about 0.001% to about 0.3% by weight of the ingestible composition. Thus, for example, EDTA can be used at about 0.0015% to about 0.002% by weight of the ingestible composition and sodium citrate at about 0.230% to about 0.260% (e.g., 0.250%) by weight of the ingestible composition.

A fluid ingestible composition can include a juice or juice concentrate and optional flavorants and/or colorants. Juices for use include fruit juices such as apple, grape, raspberry, blueberry, cherry, pear, orange, melon, plum, lemon, lime, kiwi, passionfruit, blackberry, peach, mango, guava, pineapple, grapefruit, and others known to those skilled in the art. Vegetable juices for use include tomato, spinach, wheatgrass, cucumber, carrot, peppers, beet, and others known to those skilled in the art.

The brix of the juice or juice concentrate can be in the range of from about 15 to about 85 degrees, such as about 25 to about 50 degrees, about 40 to about 50 degrees, about 15 to about 30 degrees, about 65 to about 75 degrees, or about 70 degrees. A fluid ingestible composition can have a final brix of about 2 to about 25 degrees, e.g., about 5, about 10, about 12, about 15, about 20, about 2.5, about 3, about 3.5, about 3.8, about 4, or about 4.5.

Flavorants can be included depending on the desired final flavor, and include flavors such as kiwi, passionfruit, pineapple, coconut, lime, creamy shake, peach, pink grapefruit, peach grapefruit, pina colada, grape, banana, chocolate, vanilla, cinnamon, apple, orange, lemon, cherry, berry, blueberry, blackberry, apple, strawberry, raspberry, melon(s), coffee, and others, available from David Michael, Givaudan, Duckworth, and other sources.

Colorants can also be included depending on the final color to be achieved, in amounts quantum satis that can be determined by one having ordinary skill in the art.

Rapid gelling occurs when soluble anionic fibers, such as alginate or pectin, are mixed with soluble calcium sources, particularly the calcium salts of organic acids such as lactic or citric acid. For beverage products, this reactivity prevents the administration of soluble anionic fiber and a highly soluble calcium source in the same beverage. In the present invention, this problem is overcome by administering the soluble anionic fiber and the soluble calcium source in different product components.

Solids

At least one soluble anionic fiber can be present in a solid ingestible composition in any form or in any mixtures of forms. A form can be a formed, unformed, or both. Formed forms include extruded forms, spray-dried forms, roll-dried forms, or dry-blended forms. For example, a snack bar can include at least soluble anionic fiber present as a formed food product (e.g., a crispy), at least one soluble anionic fiber in an unextruded form (e.g., as part of the bar), or both.

A formed food product can be cold- or hot-extruded and can assume any type of extruded form, including without limitation, a bar, cookie, bagel, crispy, puff, curl, crunch, ball, flake, square, nugget, and snack chip. In some cases, a formed food product is in bar form, such as a snack bar or granola bar. In some cases, a formed food product is in cookie form. In other cases, a formed food product is in a form such as a crispy, puff, flake, curl, ball, crunch, nugget, chip, square, chip, or nugget. Such formed food products can be eaten as is, e.g., cookies, bars, chips, and crispies (as a breakfast cereal) or can be incorporated into a solid ingestible composition, e.g., crispies incorporated into snack bars.

A solid form may also be a lollipop or a lolly that is made of hardened, flavored sugar mounted on a stick and intended for sucking or licking. One form of lollipop has a soft-chewy filling in the center of the hardened sugar. The soft filling may be a gum, fudge, toffee, caramel, jam, jelly or any other soft-chewy filling known in the art. The at least one multivalent cation may be in the soft-chewy center or the harnend sugar. Likewise, at least fiber may be in the soft-chewy center or the harnend sugar. A hard candy filled with a soft-chewy center is another embodiment of the present invention. This embodiment is similar to the lollipop, except it is not mounted on a stick. The soft-chewy filling may be in the center or swirled or layered with the hard sugar confection.

A cookie or mini-bar can include at least one soluble anionic fiber in an unprocessed form or in a processed (e.g., formed) form. A snack chip can include at least one soluble anionic fiber in formed form or in spray-dried form, or both, e.g., a formed soluble anionic fiber-containing chip having at least one soluble anionic fiber spray-dried on the chip.

A solid ingestible composition can include optional additions such as frostings, icings, coatings, toppings, drizzles, chips, chunks, swirls, or layers. Such optional additions can include at least one multivalent cation, at least one soluble anionic fiber, or both.

Solid ingestible compositions can provide any amount from about 0.5 g to about 10 g total soluble anionic fiber per serving, e.g., about 0.5 g to about 5 g, about 1 g to about 6 g, about 3 g to about 7 g, about 5 g to about 9 g, or about 4 g to about 6 g. For example, in some cases, about 1 g, about 2 g, about 3 g, about 4 g, about 5 g, about 6 g, about 7 g, about 8 g, or about 9 g of soluble anionic fiber per serving can be provided.

A solid ingestible composition can include at least one soluble anionic fiber at a total weight percent of the ingestible composition of from about 4% to about 50% or any value therebetween. For example, a solid ingestible composition can include at least one soluble anionic fiber of from about 4% to about 10% by weight; or about 5% to about 15% by weight; or about 10% to about 20% by weight; or about 20% to about 30% by weight; or about 30% to about 40% by weight; or about 40% to about 50% by weight.

A formed food product can be from about 0% to 100% by weight of an ingestible composition, or any value therebetween (about 1% to about 5%; about 5% to about 10%; about 10% to about 20%; about 20% to about 40%; about 30% to about 42%; about 35% to about 41%; about 37% to about 42%; about 42% to about 46%; about 30% to about 35%; about 40% to about 50%; about 50% to about 60%; about 60% to about 70%; about 70% to about 80%; about 80% to about 90%; about 90% to about 95%; about 98%; or about 99%). For example, a formed bar, cookie, or chip can be about 80% to about 100% by weight of an ingestible composition or any value therebetween.

Alternatively, an ingestible composition can include about 30% to about 55% by weight of a formed food product or any value therebetween, e.g., about 32%, about 33%, about 34%, about 35%, about 36%, about 37%, 3 about 8%, about 39%, about 40%, about 42%, about 45%, about 48%, about 50%, about 52%, or about 54% by weight of a formed food product. For example, a snack bar composition can include formed crispies in an amount of from about 32% to about 46% by weight of the snack bar.

Crispies

A formed food product, e.g., for inclusion in an ingestible composition, can be a crispy. For example, crispies that include one or more alginates and/or pectins in a total amount of about 30% to about 35% by weight can be included in a snack bar in an amount of about 32% to about 45% by weight of the snack bar. Crispies can be prepared using a fiber blend as described herein. Crispies can also include, among other things, about 52% to about 58% by weight of one or more of a rice flour, corn meal, and/or corn cone; and about 2% to about 10% of a protein isolate. Crispies can be prepared using methods known to those having ordinary skill in the art, including cold and hot extrusion techniques.

An ingestible composition or formed food product can include one or more of the following: cocoa, including flavonols, and oils derived from animal or vegetable sources, e.g., soybean oil, canola oil, corn oil, safflower oil, sunflower oil, etc. For example, a formed food product can include cocoa or oils in an amount of about 3% to about 10% (e.g., about 3% to about 6%, about 4% to about 6%, about 5%, about 6%, about 7%, or about 4% to about 8%) by weight of the formed food product.

One embodiment of the present invention is a stable two-phase product having at least one soluble anionic fiber and at least one multivalent cation in the same product, but formulated so that the soluble anionic fiber and multivalent cation do not react during processing or prior to ingestion, but react following ingestion as a standard multivalent cation-anion fiber reaction. One product design includes a jam phase center and a crisp baked solid phase outside the fluid jam phase. One embodiment places the soluble anionic fiber in the jam phase and places the multivalent cation in the baked dough phase. However, it has been found that the stability of this embodiment is less than optimal from an organoleptic standpoint. That is, it provided a solid, rubberlike jam phase instead of pleasant texture due to the migration of the multivalent cation from the baked dough phase.

Adding the soluble anionic fiber to the baked dough phase and the multivalent cation to the jam phase, which provides a cookie that reduces the water activity of the fiber-containing phase that restricted fiber so that it was prevented from reacting with the multivalent cation. The placement of the multivalent cation into a postbake, medium water activity filler, e.g., the jam phase, allowed the cation to be formulated in the product with an acceptable organoleptic profile and an inability to react with fiber even if minor migration occurs.

The water activities of both components can be further adjusted to deliver a product with not only restrictive reaction in place but acceptable eating qualities and the right characteristics needed to for ease of manufacturing.

Types of salts tested include calcium fumarate, tricalcium phosphate, dicalcium phosphate dihydrate and calcium carbonate. The gram weight tested will vary depending on the salt type due to its characteristic calcium load. The piece weight of the product under discussion has been about 13 to about 20 g, with each piece delivering 50 to about 75 kcal.

BENEFAT® is a family of triglyceride blends made from the short and long chain fatty acids commonly present in the diet. It is the uniqueness of these fatty acids that contribute to the range's reduced calorie claim. BENEFAT® products are designed to replace conventional fats and oils in dairy, confectionery and bakery products, giving full functionality with significantly reduced energy and fat content. BENEFAT® is the Danisco trade name for SALATRIM, the abbreviation for short and long-chain triglyceride molecules. The short-chain acids (C₂-C₄) may be acetic, propionic, butyric or a combination of all three, while the long-chain fatty acid (C₁₆-C₂₂) is predominantly stearic and derived from fully hardened vegetable oil. Unlike other saturated fatty acids, stearic acid has a neutral effect on blood cholesterol. BENEFAT® is also free of trans fatty acids and highly resistant to oxidation. Compared to the 9 calories per gram of traditional fat, BENEFAT® contains just 5 calories per gram (US regulation) or 6 calories per gram (EU regulation), at the same time giving foods a similar creamy taste, texture, and mouthfeel as full-fat products. Metabolisation upon consumption occurs in much the same way as with other food components.

A preferred product features include about 500 to about 1500 mg of alginate are present, the multivalent cation is calcium wherein about 50 to about 500 mg of elemental calcium are delivered. The product has low calories between about 50 to about 100 calories and is a cookie with a jam filling.

The soluble anionic fiber can be provided in one beverage component, and a soluble calcium source can be provided in a second beverage component. The first component and the second component are provided separately to the user in a bottle or cup, and the user consumes the two components concurrently or sequentially.

The soluble anionic fiber may be delivered in a beverage component and a soluble calcium source may be provided separately in a solid edible component. The fluid fiber component and the solid calcium-containing component are consumed concurrently or sequentially.

The soluble anionic fiber component may be provided in a solid edible component and the soluble calcium source may be provided separately in a fluid component. The fluid calcium-containing component and the solid fiber-containing component are consumed concurrently or sequentially.

The soluble anionic fiber component and the soluble calcium source are both provided in solid edible components. The components may be provided in the form of separate items for consumption, or both components may be combined in a single solid form for consumption. This single solid form may contain the soluble anionic fiber in one phase, such as a layer or filling, and the calcium source may be provided in a separate phase, such as a layer or filling. Alternatively, the fiber and calcium source may be intimately mixed in the same solid form.

The ingestible composition useful in the present invention can be provided in any package, such as enclosed in a wrapper or included in a container. An ingestible composition can be included in an article of manufacture. An article of manufacture that includes an ingestible composition described herein can include auxiliary items such as straws, napkins, labels, packaging, utensils, etc.

An article of manufacture can include a source of at least one multivalent cation. For example, a source of at least one multivalent cation can be provided as a fluid, e.g., as a beverage to be consumed before, during, or after ingestion of the ingestible composition. In other cases, at least one multivalent cation can be provided in a solid or gel form. For example, a source of at least one multivalent cation can be provided in, e.g., a jelly, jam, dip, swirl, filling, or pudding, to be eaten before, during, or after ingestion of the ingestible composition. Thus, in some embodiments, an article of manufacture that includes a cookie or bar solid ingestible composition can also include a dip comprising a source of at least one multivalent cation, e.g., into which to dip the cookie or bar solid ingestible composition.

Also provided are articles of manufacture that include a fluid ingestible composition. For example, a fluid ingestible composition can be provided in a container. Supplementary items such as straws, packaging, labels, etc. can also be included. Alternatively, the soluble anionic fiber may be included in a beverage and the multivalent cation may be provided inside, outside or both of a straw or stirring stick. In some cases, at least one multivalent cation, as described below, can be included in an article of manufacture. For example, an article of manufacture can include a fluid ingestible composition in one container and a source of multivalent cations in another container. Two or more containers may be attached to one another.

Methods of Reducing Caloric Consumption

A soluble anionic fiber (such as alginate and pectin) is administered concurrently with a multivalent cation source such as a water-soluble calcium salt to reduce food intake. Continued use of these compositions by individuals in need of weight loss will result in a cumulative decrease in caloric consumption, which will result in weight loss or diminished weight gain. Although not wishing to be bound by theory, the inventors hypothesize that the multivalent cation calcium ions of the soluble calcium source cross link the carboxylate groups on the fiber molecules, resulting in the formation of highly viscous or gelled materials. This gelling effect increases the viscosity of the gastric and intestinal contents, slowing gastric emptying, and also slowing the rate of macro-nutrient, e.g., glucose, amino acids, fatty acids, and the like. These physiological effects prolong the period of nutrient absorption after a meal, and therefore prolong the period during which the individual experiences an absence of hunger. The increased viscosity of the gastrointestinal contents, as a result of the slowed nutrient absorption, also causes a distal shift in the location of nutrient absorption. This distal shift in absorption may trigger the so-called “ileal brake” and the distal shift may also cause in increase in the production of satiety hormones such as GLP-1 and PYY.

Provided herein are methods employing the ingestible compositions described herein. For example, a method of facilitating satiety and/or satiation in an animal is provided. The method can include administering an ingestible composition to an animal. An animal can be any animal, including a human, monkey, mouse, rat, snake, cat, dog, pig, cow, sheep, horse, or bird. Administration can include providing the ingestible combination either alone or in combination with other meal items. Administration can include co-administering, either before, after, or during administration of the ingestible composition, a source of at least one multivalent cation, such as, calcium, or a sequestered source of calcium, as described herein. At least one multivalent cation can be administered within about a four-hour time window flanking the administration of the ingestible composition. For example, a source of calcium, such as a solution of calcium lactate, can be administered to an animal immediately after the animal has ingested a fluid ingestible composition as provided herein. Satiety and/or satiation can be evaluated using consumer surveys (e.g., for humans) that can demonstrate a statistically significant measure of increased satiation and/or satiety. Alternatively, data from paired animal sets showing a statistically significant reduction in total caloric intake or food intake in the animals administered the ingestible compositions can be used as a measure of facilitating satiety and/or satiation.

As indicated previously, the ingestible compositions provide herein can hydrate and gel in the stomach and/or small intestine, leading to increased viscosity in the stomach and/or small intestine after ingestion. Accordingly, provided herein are methods for increasing the viscosity of stomach and/or small intestine contents, which include administering an ingestible composition to an animal. An animal can be any animal, as described above, and administration can be as described previously. Viscosity of stomach contents can be measured by any method known to those having ordinary skill in the art, including endoscopic techniques, imaging techniques (e.g., MRI), or in vivo or ex vivo viscosity measurements in e.g., control and treated animals.

The inventors have found that a product containing an anionic fiber (alginate) in combination with a multivalent cation (calcium) is effective, in comparison to a similar composition lacking alginate, in promoting weight loss in individuals on a low intensity weight loss program. The low intensity weight loss program involved a focus on monitoring food consumption and promoting exercise, but did not involve the extensive or uncomfortable dieting regimens of many weight loss programs (such programs are frequently unsuccessful in achieving weight loss because individuals fail to comply because of the hunger and discomfort arising from the dieting regimen). The inventors have found that compositions containing effective amounts of a combination of soluble anionic fiber and multivalent cation, when consumed in the context of a low intensity weight loss program, resulted in a caloric deficit of about 150 kcal to about 300 kcal per day.

Without being bound by theory, the inventors believe that consumption of compositions containing weight loss effective amounts of a combination of a soluble anionic fiber and a multivalent cation, in the context of a low intensity weight loss program, is an especially effective means of promoting weight loss in an individual desiring weight loss. The effects of the combination of anionic fiber and multivalent cation in increasing the viscosity of the gastrointestinal contents and slowing nutrient absorption prolong satiety and reduce appetite in individuals consuming these compositions, thereby aiding their effort to comply with this low intensity weight loss program. The consumption of these compositions is especially effective in achieving caloric deficits of about 150 to about 300 kcal per day, when consumed in the context of a low intensity weight loss program.

Additionally, with being bound by theory, the inventors believe that caloric deficits of greater than about 350 kcal to about 1000 kcal per day, particularly greater than about 500 kcal per day, will cause food cravings and hunger sensations of such magnitude in many individuals so as to overwhelm the food intake reducing properties of the inventive compositions.

Weight Loss/Weight Maintenance Programs

Any weight loss/weight maintenance program can be used in the present invention. It is preferred that the weight loss/weight maintenance program include an exercise component. Weight loss programs include meal planning, meal replacement, portion control, exercise, caloric dilution, cognitive modification, group or individual counseling, coaching, or support, or combinations thereof. Examples of currently popular weight loss/weight management programs include the SOUTH BEACH DIET®, the ATKINS DIET®, NUTRITSYSTEM®, JENNY CRAIG®, MEDIFAST®, WEIGHT WATCHERS®, BODY FOR LIFE®, Step Diet, and the like.

The SOUTH BEACH DIET® includes the following phases:

Phase 1: The South Beach Diet begins with a restricted two-week induction phase where most carbohydrates (such as, rice, pasta, and breads) must be avoided. There are three meals a day and snacks which are eaten until hunger is satisfied. Meats, shellfish, chicken, turkey, and fish can be eaten-along with nuts, cheese (fat-free), eggs, salads, and vegetables.

Phase 2: The second phase includes specific meal plans and recipes. It sparingly reintroduces some of the foods avoided in Phase 1. This length of time on this phase is dependent on the individual's goals.

Phase 3: The third phase is about living the lifestyle more than a phase. This phase is about eating healthy and weight maintenance

NUTRISYSTEM® is a portion-controlled weight loss program that provides on-line analysis to calculate an individual's calorie requirements. From this, meal plans can be calculated and the company will ship all meals to an individual.

The ATKINS DIET® is diet that severely restricts carbohydrate intake. Carbohydrates sources such as foods with sugar, bread, cereal, some starchy vegetables and pasta are avoided. Weight loss on the ATKINS DIET® is based on the premise that the main source of energy for humans is carbohydrates. When a human is carbohydrate challenged, the body must use another source of energy. The next energy source for the body is stored body fat. Once the body is using fat as an energy source, the body is said to be in ketosis. Another premise is that carbohydrates stimulate the creation of insulin. Insulin converts excess carbohydrates to fat. Thus, the less carbohydrates available, the less insulin produced and the less fat created.

MEDIFAST® is a fast weight loss plan using meal replacements and regular food. The program has been prescribed by doctors for many years (particularly for obese people). This 5 and 1 plan is made up of 5 meal replacements per day, including shakes, bars, soups, oatmeal, and puddings. One meal per day is a “lean and green” meal—a small portion of lean meat and up to 2 cups of salad or vegetables. Individuals eat every 2-3 hours and must drink a minimum of 64 oz of fluid (water) per day. Other beverages can be consumed in addition to this.

WEIGHT WATCHERS® is a portion control and exercise plan. The core plan includes eating portions from a list of healthy foods from all the food group, having an occasional treat, and exercise.

The JENNY CRAIG® weight management program is a portion-controlled diet plan based around the traditional United States dietary guidelines (e.g., USDA food pyramid). It is a calorie controlled program where all meals are shipped to the individual. The program involves visiting a JENNY CRAIG® center for weigh-ins, and consulting one-on-one with one of their weight loss consultants. A fitness and exercise component is also part of the program.

The BODY FOR LIFE® diet includes 6 meals per day. Portion size is emphasized rather than calorie counts. A typical meal might include one portion of protein, and one portion of carbohydrate. Cheating is allowed one day each week. The exercise component includes 20 minutes 3 times per week of aerobic exercise, and lifting weights for 3 times a week (45 minutes per session).

The Step Diet has six components: 1) prepare for weight management, 2) stop gaining weight, 3) Set realistic goals, 4) make small changes to an individual's daily routine, e.g., take the stairs instead of an elevator, 5) find energy balance point that increases exercise to make up for the drop in metabolism, and 6) plan for lifelong success. For example, get as much walking and physical activity in as an individual can and have the individual go back and adjust how much they eat. The more an individual can walk, the more the individual can eat.”

Also provided are methods for promoting weight loss by administering an ingestible composition as provided herein to an animal. Administration can be as described previously. The amount and duration of such administration will depend on the individual's weight loss needs and health status, and can be evaluated by those having ordinary skill in the art. The animal's weight loss can be measured over time to determine if weight loss is occurring. Weight loss can be compared to a control animal not administered the ingestible composition.

The following examples are representative of the invention, and are not intended to be limiting to the scope of the invention.

### EXAMPLES

### Example 1

A cookie having a solid phase, e.g., a baked dough phase, containing a soluble anionic fiber blend and a fluid phase, e.g., jam phase containing a soluble calcium source deposited in the baked dough phase was produced.

The baked dough phase was prepared by adding BENEFAT® and lecithin to a premix of flour, cellulose, egg white, salt, leavening and flavors in a Hobart mixer and creaming by mixing at low speed for about 1 minute followed by high speed for about 2 minutes. The liquids were added to creamed mixture and blended at medium speed for about 2 minutes.

The fiber blend used contained about 46% sodium alginate LBA (ISP, San Diego, Calif.), about 39.6% sodium alginate GHB (ISP), and about 14.4% pectin (USP-L200, Kelco, San Diego, Calif.).

The fiber blend and glycerin were added to a separate bowl and combined. This combined fiber/glycerin material was added to the other ingredients in the Hobart mixer and was mixed on medium speed for about 1 minute. The resulting dough was then sheeted to desired thickness on a Rhondo sheeter and a dough pad measuring about 3 inched by about 6 inches was created.

The jam phase was prepared by adding a premixed BENEFAT®/calcium source mixture to the jam base and mixed until uniformly mixed. A predetermined amount of the jam was then added onto the top surface of the cookie dough pad. The dough pad edges were wetted and sealed. Bars were baked at 325° F. for about 9 minutes, cut, cooled and the resulting cookies were individually packaged. The total caloric value of each cookie was about 50 kcal.

Dough Phase

| | % Dough | % Total |
|---|---|---|
| Ingredient | Phase | Formulation |
| flour - all purpose | 29.140 | 12.165 |
| cellulose, solka floc - International | 6.980 | 2.914 |
| Fiber Corp. | | |
| Powder egg white | 0.580 | 0.242 |
| salt (NaCl) | 0.200 | 0.083 |
| sodium Bicarbonate Grade #1 | 0.510 | 0.213 |
| cookie Dough Flavor | 0.170 | 0.071 |
| BENEFAT | 2.060 | 0.860 |
| Lecithin | 0.640 | 0.267 |
| polydextrose litesse 70% syrup, Ultra | 15.870 | 6.625 |
| Water | 11.830 | 4.939 |
| Liquid vanilla flavor | 0.280 | 0.117 |
| sucralose, 25% liquid. | 0.090 | 0.038 |
| potassium sorbate | 0.250 | 0.104 |
| alginate fiber blend | 17.400 | 7.264 |
| glycerine, optima 99.7% USP | 14.000 | 5.845 |
| | 100.000 | 41.70 |

Jam Phase:

| | % Jam | % Total |
|---|---|---|
| Ingredient | Phase | Formulation |
| BENEFAT | 21.100 | 12.291 |
| calcium fumarate trihydrate | 11.000 | 6.408 |
| reduced calorie strawberry filling | | |
| (SMUCKERS) | 67.900 | 39.553 |
| | 100.000 | 58.25 |

Control

Dough Phase:

| | % Dough | % Total |
|---|---|---|
| Ingredient | Phase | Formulation |
| Flour - all purpose | 29.140 | 12.530 |
| cellulose, solka floc - International | 6.980 | 3.001 |
| Fiber Corp. | | |
| powder egg white | 0.580 | 0.249 |
| salt (NaCl) | 0.200 | 0.086 |
| sodium bicarbonate Grade #1 | 0.510 | 0.219 |
| cookie dough flavor | 0.170 | 0.073 |
| BENEFAT | 19.450 | 8.364 |
| Lecithin | 0.640 | 0.275 |
| polydextrose litesse 70% syrup, Ultra | 15.870 | 6.824 |
| Water | 11.830 | 5.087 |
| Liquid vanilla flavor | 0.280 | 0.120 |
| sucralose, 25% liquid. | 0.090 | 0.039 |
| potassium sorbate | 0.250 | 0.108 |
| alginate fiber blend | 0.000 | 0.000 |
| glycerine, Optim 99.7% USP | 14.000 | 6.020 |
| | 100.000 | 43.00 |

Jam Phase:

| | % Jam | % Total |
|---|---|---|
| Ingredient | Phase | Formulations |
| BENEFAT | 32.100 | 19.260 |
| reduced calorie | | |
| strawberry filling | | |
| (SMUCKERS) | 67.900 | 40.740 |
| Total | 100.000 | 60.00 |

Measurement of Intestinal Viscosity

Fully grown female Yucatan minipigs (Charles River Laboratories, Wilmington, Mass.), weighing about 90 kg, were fitted with indwelling silicone rubber sample ports (Omni Technologies, Inc., Greendale, Ind.) implanted in a surgically created dermal fistula at the ileocecal junction. The sample ports were sealed by a removable cap. These ports permit removal of samples of digesta as it passes from the ileum to the cecum. Additional details of this procedure were presented in B. Greenwood van-Meerveld et al., Comparison of Effects on Colonic Motility and Stool Characteristics Associated with Feeding Olestra and Wheat Bran to Ambulatory Mini-Pigs, Digestive Diseases and Sciences 44:1282-7 (1999), which is incorporated herein by reference.

Three Yucatan minipigs with the fistulas described above were housed in individual stainless steel pens in a windowless room maintained on a cycle of 12 hours of light and 12 hours of dark. They were conditioned to consume low fiber chow (Laboratory Mini-Pig Diet 5L80, PMI Nutritional International, Brentwood, Mo.). This chow contains about 5.3% fiber. The pigs were fed once each day, in the morning. Water was provided ad libitum throughout the day.

Samples were taken from the ileal sample port immediately after feeding, and then at about 30 minute intervals for about 300 minutes. The volume of sample collected was about 50 to 130 ml. All samples were assayed for viscosity within 30 minutes after collection.

Samples of digesta were collected in sealed plastic containers. Viscosity of the digesta was measured with a Stevens QTS Texture Analyzer (Brookfield Engineering, Inc., Middleboro, Mass.). This instrument measures the relative viscosity of digesta by a back extrusion technique. The instrument was comprised of a stage plate, a 60 cm vertical tower, a mobile beam and a beam head that contains a load-cell. During back extrusion, the beam descends at a constant rate, and the force required to back extrude the sample was recorded over time. The sample containers were 5 cm deep spherical aluminum cups with an internal diameter of about 2.0 cm. The volume of the cup was about 20 ml. The spherical probe consists of a 1.9 cm Teflon ball mounted on a 2 mm threaded rod which was attached to the mobile beam. The diameters of the sample cup and probe allow for a wide range of viscosity (liquid to solid digesta) to be measured without approaching the maximum capacity of the rheometer (25 kg/peak force). During each test, the beam thrusts the probe into the test sample at a constant rate (12 cm/second) for a 2 cm stroke, forcing the sample to back-extrude around the equatorial region of the probe. The peak force for back extrusion at a controlled stroke rate was proportional to the viscosity of the sample. At each time point, 2-6 samples from each pig were tested, and the mean peak force was calculated and recorded.

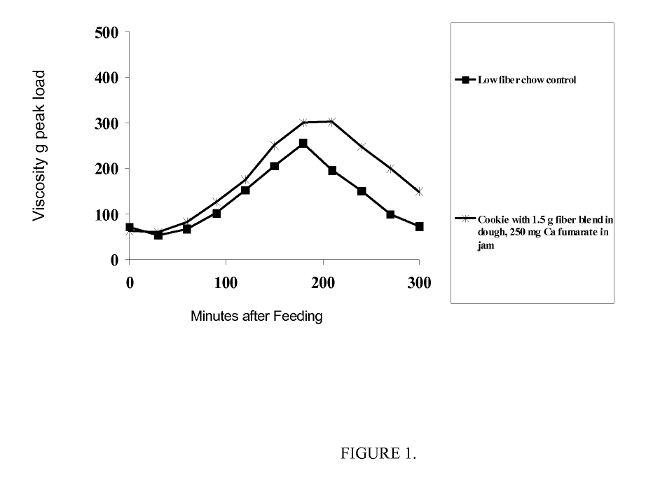

The test for effects of fiber containing cookies on viscosity was performed by providing each pig with its daily ration of low fiber chow (1400 g). Before feeding, one cookie was gently broken into four to six pieces and mixed into the chow. The animals have unlimited access to water during and after feeding. The effect of the cookie of this example containing fiber and calcium on intestinal viscosity was shown in FIG. 1. Each treatment was provided to each of three pigs on three separate days to yield nine replicates for each sample. Each point plotted in FIG. 1 is the mean of these nine determinations. The fiber and calcium containing cookie produced viscosities significantly greater than those produced by control chow (p<0.05, as measured by a two-tailed t-test) at the time points from 210 minutes through 300 minutes.

### Example 2

A study to evaluate the effects of soluble fiber and calcium on food intake was performed by the following procedure.

The study was a within-subjects design with 30 participants completing three one week treatment periods, with a washout period of one week between treatment periods. Treatment order was counterbalanced to have five subjects randomly assigned to each of six possible treatment sequences. Subjects in each treatment period consumed a test beverage at breakfast and after lunch (mid-afternoon). In one treatment period, subjects consumed a placebo beverage without fiber. In two treatment periods, the test beverage contained a blend of soluble fibers of one of the following compositions:

| | 2.8 g Fiber | 1.0 g Fiber | Placebo |
|---|---|---|---|
| Ingredient | % | % | % |
| Water | 95.470 | 96.400 | 97.010 |
| Trisodium citrate dihydrate | 0.250 | 0.250 | 0.250 |
| LBA alginate (ISP) | 0.640 | 0.210 | 0.000 |
| GHB alginate (ISP) | 0.550 | 0.180 | 0.000 |
| USP L200 pectin (Kelco) | 0.200 | 0.066 | 0.000 |
| Apple juice concentrate | 2.300 | 2.300 | 2.300 |
| EDTA | 0.002 | 0.002 | 0.002 |
| Sucralose | 0.011 | 0.011 | 0.011 |
| Malic acid, granular | 0.200 | 0.200 | 0.200 |
| Red 40, 10% solution | 0.001 | 0.001 | 0.001 |
| Flavor | 0.380 | 0.380 | 0.380 |
| Total | 100.000 | 100.0001 | 100.000 |

The fiber drinks were consumed with a separate beverage containing calcium lactate (not more than 500 mg elemental calcium per serving). The placebo was taken with a second placebo beverage matched for flavor and calories, but without calcium lactate. The test drink containing calcium lactate or corresponding placebo had the following composition:

| | Calcium Placebo | Calcium Free Placebo |
|---|---|---|
| Ingredient | % | % |
| Water | 96.430 | 99.846 |
| Calcium lactate | 3.065 | 0.000 |
| Malic acid | 0.330 | 0.330 |
| Sucralose | 0.050 | 0.020 |
| Yellow #5, 1% solution | 0.007 | 0.007 |
| Red #40, 1% Solution | 0.0069 | 0.0069 |
| Flavor | 0.110 | 0.110 |
| Total | 100.000 | 100.000 |

Subjects in the study were premenopausal women selected without regard to racial or ethnic background. Eligible women had to be between 20 and 40 years of age, non-smokers, and overweight or obese (body mass index, or BMI, of 25-35 kg per square meter).

Test Sessions and Experimental Measurements

Test sessions occurred on the first and seventh day of the use of each experimental period. The night before the sessions, subjects consumed an evening meal of their own choosing that was replicated the night before each test session. Test sessions began between 7:00 and 9:00 AM. Subjects first completed a short questionnaire to ensure they had consumed the evening meal, and had not been ill in the previous week. Immediately before a standardized breakfast meal (choice of bagel or raisin bran cereal) they were asked to consume a fiber test beverage within a three minute interval, which consists of the first part of the test beverage (fiber or placebo) first, immediately followed by the second part of the test beverage calcium or placebo). They were then served the standard breakfast. They returned to the lab for lunch 4-5 hours later, and dinner 9-10 hours later. They were provided with a portable cooler containing the test beverage (fiber or placebo beverage, and the calcium beverage or calcium-free placebo beverage), and a bottle of water. They were instructed to consume the test beverage 2½ hours after the completion of lunch and not to consume any food during the day except the test meals provided, the test beverages, and the bottled water.

At the test sessions, lunch and dinner were provided as buffet-style meals. Subjects were also provided snacks for consumption during the evening. They were told to consume as much of the snacks as they desired. Lunch and dinner servings of each individual food were weighed to the nearest 0.1 g before and after consumption to determine caloric and macronutrient intake. Evening snacks were returned to the test site to determine food consumption.

Subjects were asked to consume 14 test drinks during each week of the three week long experimental periods. On Day 1, as mentioned above, they drank one two-part test beverage before breakfast, and one 2.5 hours after lunch. Additionally, on the first test day they were provided with five refrigerated test beverages (5 first part and 5 second part) to take home. They were instructed to consume one test beverage, which was one first part followed by one second part, before breakfast, and another test beverage about 3½ hours after lunch each day on the second through sixth days. Subjects returned to the laboratory on the seventh day to repeat the procedure of the first day.

Data Analysis

Data were analyzed using the Statistical Analysis System (SAS Version 8.2, Cary, N.C.). The mixed model procedure was used to test for treatment differences, with treatment condition (low fiber, high fiber, and placebo), day (1 or 7) and the interaction of condition and day entered into the statistical models. The effects of treatment session was also tested as a covariate and kept in the final model when found to be significant. The endpoint measurements included the total daily energy and macronutrient content of foods consumed, as well as at each individual meal (breakfast, lunch, dinner, and evening snack).

Consumption of the two different fiber containing beverages (1 g and 2.8 g per serving) resulted in a trend toward reduction in total calorie intake measured over the 24 hour period beginning with the morning beverage.

Effect of Fiber Beverages on Total Calorie

| | Mean Kcal | Standard | P value vs. |
|---|---|---|---|
| Condition | Intake | Error | placebo |
| Placebo | 2634 | 109 | 0.17 |
| 1 g fiber beverage | 2512 | 110 | 0.17 |
| 2.8 g fiber beverage | 2510 | 109 | |

Consumption of both the fiber containing beverages (1 g and 2.8 g per serving) resulted in a significant decrease in food consumption at dinner, as shown below.

Effect of Fiber Beverages on Caloric Intake at Dinner

| | Mean Kcal | Standard | P value vs. |
|---|---|---|---|
| Condition | Intake | Error | placebo |
| Placebo | 765 | 37 | |
| 1 g fiber beverage | 689 | 37 | 0.039 |
| 2.8 g fiber beverage | 678 | 37 | 0.016 |

The 1 g fiber beverage reduced dinner food intake by an average of 76 kcal, and the 2.8 g beverage provided a reduction of 87 kcal. The P values, determined by a post-hoc Tukey's analysis, indicated that these results were statistically significant (p<0.05).

Further analysis of the nutrient composition of the individual foods consumed indicated that the consumption of the fiber beverages was associated with a significant reduction in the intake of carbohydrates at dinner, as shown below.

Effect of Fiber Beverages on Carbohydrate
Caloric Intake at Dinner

| | Mean Carbohydrate | Standard | P value vs. |
|---|---|---|---|
| Condition | Kcal Intake | Error | placebo |
| Placebo | 379 | 21 | |
| 1 g fiber beverage | 329 | 21 | 0.007 |
| 2.8 g fiber beverage | 324 | 21 | 0.003 |

The 1 g beverage reduced carbohydrate intake at dinner by 50 kcal, and the 2.8 g beverage provided a 55 kcal reduction. The reduction in carbohydrate intake at both levels was statistically significant (p<0.01).

The fiber beverages also reduced total daily food intake, as shown below.

Effects of Fiber Beverages on Daily Caloric Intake

| | Mean Kcal | Standard | P value vs. |
|---|---|---|---|
| Condition | Intake | Error | placebo |
| Placebo | 1353 | 64 | |
| 1 g fiber beverage | 1261 | 64 | 0.026 |
| 2.8 g fiber beverage | 1264 | 64 | 0.033 |

The 1 g fiber beverage reduced overall food intake on the test day by an average of 92 kcal, and the 2.8 g beverage provided a reduction of 89 kcal. The P values, determined by a post-hoc Tukey's analysis, indicated that these results were statistically significant (p<0.05). These results indicated the absence of compensatory eating that could have occurred in response to the reduced dinner caloric intake.

### Example 3

Subjects were recruited from a group of individuals who completed a 16-week weight loss trial. That weight loss trial involved an intensive weight loss regimen including a recommendation to decrease caloric intake by 1000 kcal per day (compared to intake prior to beginning the trial), increasing physical activity by 500 steps per day in each week of the trial (as measured by a pedometer), and behavioral intervention. After completing this initial weight loss trial, subjects volunteered to participate in an additional weight loss trial involving a different treatment regimen. This regimen was a fiber containing nutritional bar (or placebo) consumed twice per day at times selected by the subject. The inclusion and exclusion criteria for these subjects were presented in the following table.

| Inclusion Criteria: | Exclusion Criteria: |
|---|---|
| Age: 20-45 years old | Par-Q showing underlying disease that |
| | would require monitoring physical |
| | activity |
| Gender: male or female | Irritable bowel syndrome |
| BMI of 27-35 | Diabetes |
| Healthy | Gastrointestinal conditions |
| | Malabsorption syndromes |
| | Weight loss of more than 10 pounds in 3 |
| | months prior to first weight loss trial |
| | Eating disorders (i.e., binge eating, |
| | purging) |
| | Currently taking medications that affect |
| | appetite |
| | Pregnant or lactating women |

The fiber bar in the second weight loss trial was an unbaked, formed bar made of formed, crunchy bits (or crispies) with 3 grams of alginate (Manugel DPB), agglomerated with rolled oats, raisins and dried cranberries (for color and texture) using a syrup containing calcium phosphate (300 mg elemental calcium), and formed into bars. Each 30-g bar contains 100 kcal. Placebo bars were matched for taste, texture, and calcium content, but contained no alginate. The bar composition allowed the calcium and alginate to be kept separate in the same form until it was consumed (the fiber was present in the crispies, and the calcium was in the syrup that held the crispies together). Bars were designated as “A” (placebo) or “B” (alginate), but neither the test site nor the subject knew the identity of the bars. The compositions of both of the bars were shown in the following tables.

To produce a batch of crisps, the ingredients are dry blended in a small ribbon blender. The resulting dry blend is transferred using a feeder, e.g., a K-Tron loss-in-weight feeder, into the hopper of an extruder e.g., a Buhler Twin Screw Extruder configured with at least one heating unit, e.g., two Mokon barrel-heating units. Water is added as steam to the dry blend using a barrel injection system. A second liquid can also be introduced at variable rates by another injector the barrel. The blend is then mixed and cooked in the extruder. The hot pressurized product stream is forced through a die for expansion, cut, and then conveyed by vacuum or mechanical conveying to a fluid bed drier, e.g., Buhler fluid bed drier, and dried to the desired moisture content. The fluid bed drier can dry about 50 to about 100 kg/hour at temperatures from about 20° to about 110° C.

Alginate Containing Bars:

Alginate Crisps

Formula # 5981-15-15

| | Ingredients | % |
|---|---|---|
| 1 | Rice Flour (PGP International) | 56.50 |
| 2 | Alginate DPB (ISP) | 31.50 |
| 3 | Whey Protein Isolate BiPro (Davisco) | 4.00 |
| 4 | Corn Starch (Cargill) | 3.00 |
| 5 | Fractionated Canola Oil (Cargill Solo 1000) | 5.00 |
| | Total | 100.00 |

Bars Containing Crisps:

| # | Ingredients | % in Bar |
|---|---|---|
| 1 | High Maltose Corn Syrup (Cargill) | 15.64 |
| 2 | MFCS (Cargill) | 5.81 |
| 3 | Dark Molasses (Christian Hansen) | 1.02 |

Step 1: Weigh and cook all above liquid at 160° F.

| | | |
|---|---|---|
| 4 | Maltodextrin DE 7.5 (Cargill) | 1.53 |
| 5 | Fructose (Univar USA) | 3.57 |
| 6 | Dicalcium Phosphate Anhydrous (Chemische | 3.37 |
| | Fabrik Budenheim) | |
| 7 | Citric Acid (Cargill) | 0.07 |

Step 2: Add all dry ingredients, cook Brix to 88%

| | | |
|---|---|---|
| 8 | Canola Oil (Cargill) | 0.68 |
| 9 | Vanilla Flavor FJ1678 (Unger) | 0.51 |
| 10 | Cranberry Flavor (Comax) | 0.17 |
| | Total | 32.37 |

Step 3: Add flavors and oil, mix well and cook gently, check
Brix to 87%

| | | |
|---|---|---|
| 11 | Test Crisps | 32.00 |
| 12 | Rolled Oats, Thick Rolled #3 (Grain Millers) | 18.00 |
| 13 | Raisins (Van Drunen) | 4.00 |
| 14 | Cranberry Halves (Van Drunen) | 12.00 |

Step 4: Add syrup to dry ingredients, mix quickly
Step 5: Transfer the mass to a pan, roll flat, cool for a minimum of
15 minutes
Step 6: Cut to L 4.0″, W 1.55″ and H 0.8″, then wrap

| | |
|---|---|
| Total | 100.00 |

Placebo:

Placebo Crisps:

Formula # 5981-15-25

| | Ingredients | % |
|---|---|---|
| 1 | Rice Flour (PGP International) | 88.00 |
| 2 | Whey Protein Isolate BiPro (Davisco) | 4.00 |
| 3 | Corn Starch (Cargill) | 3.00 |
| 4 | Fractionated Canola Oil (Cargill Solo 1000) | 5.00 |
| | Total | 100.00 |

Bars Containing Placebo Crisps:

| # | Ingredients | % in Bar |
|---|---|---|
| 1 | High Maltose Corn Syrup (Cargill) | 15.64 |
| 2 | HFCS (Cargill) | 5.81 |
| 3 | Molasses, Dark (Christian Hansen) | 1.02 |

Step 1: Weigh and cook all above liquid at 160° F.

| | | |
|---|---|---|
| 4 | Maltodextrin DE 7.5 (Cargill) | 1.53 |
| 5 | Fructose (Univar USA) | 3.57 |
| 6 | Dicalcium Phosphate Anhydrous) (Chemische | 5.00 |
| | Fabrik Budenheim) | |
| 8 | Citric Acid (Cargill) | 0.07 |

Step 2: Add all dry ingredients, cook Brix to 88%

| | | |
|---|---|---|
| 9 | Canola Oil (Clear Valley) | 0.68 |
| 10 | Vanilla Flavor FJ1678 (Unger) | 0.51 |
| 11 | Cranberry Flavor (Van Drunen) | 0.17 |
| | Total | 34.00 |

Step 3: Add flavors and oil, mix well and cook gently, check
Brix to 87%

| | | |
|---|---|---|
| 12 | Test Crisps (5981-15-15) | 32.00 |
| 13 | Rolled Oats, Thick Rolled #3 (Grain Millers) | 13.00 |
| 14 | Whole Almonds (Paramount Farms) | 5.00 |
| 15 | Raisins (Van Drunen) | 8.00 |
| 16 | Cranberry Halves (Van Drunen) | 8.00 |

Step 4: Add syrup to dry ingredients, mix quickly
Step 5: Transfer the mass to a pan, roll flat, cool for a minimum of
15 minutes
Step 6: Cut to L 4.0″, W 1.55″ and H 0.8″, then wrap

| | |
|---|---|
| Total | 100.00 |

The subjects were randomized to receive one of the two treatments.

Subjects were asked to consume two bars per day, when hungry, for a 12-week period, to track their caloric intake and physical activity levels by using a diary, and return to the clinic monthly for weigh-ins. Consciousness of exercise level and motivation to increase exercise was promoted by providing subjects with pedometers to record the number of steps taken per day. This was by intent a low intensity weight loss counseling program, and no other intervention or training was provided during the twelve-week trial. Results of the trial were presented in the following table.

Change in Weight (Pounds) at Indicated Time (versus Baseline)

| | 4 Weeks | 8 Weeks | 12 Weeks |
|---|---|---|---|
| Active (alginate) | −2.1 | −3.1 | −3.7 |
| Placebo | −0.6 | −1.1 | −0.2 |

Subjects consuming the test bar containing alginate and calcium continued to loose weight during the 12-week monitoring period relative to those consuming a control bar (placebo; calcium only). The group consuming the alginate bar lost significantly more weight than the placebo group at every weigh-in. The trend to weight loss remained consistent for the test bar group throughout the trial, whereas the control bar group continued the weight loss trend at a significantly lower rate through week 8 (second weigh-in period), and then returned very nearly to baseline weight. There was no significant difference in exercise levels between the two groups (as determined by the pedometer step counts).

The group receiving the active bar product lost an average of 3.7 pounds, or 1.68 kilograms over the 12 week study. In humans participating in a long-term weight loss program, a gram of weight loss is generally considered to require a caloric deficit of 8 kcal; this caloric deficit can be achieved either by reduced food consumption or increased energy expenditure through exercise. Therefore the cumulative caloric deficit over the 12-week study for the individuals receiving the active bar was about 13,500 calories, or a mean daily deficit of 160 kcal.

Considering only the weight loss observed over the first eight weeks of treatment, the cumulative caloric deficit over eight weeks was about 11,300 kcal, or a mean daily deficit of about 201 kcal per day.

Considering only the weight loss observed over the first four weeks of the treatment, the cumulative caloric deficit over four weeks was about 7,600 kcal, or about 270 kcal per day.

The weight loss program of this example was of relatively moderate intensity, and did not require participants to undergo extreme fasting or dieting, or extensive or intensive exercise programs. Without being bound by theory, the inventors believe that the relatively modest nature of this weight loss regimen permitted improved performance of the fiber-containing bar product that was administered to facilitate weight loss. In particular, this program did not involve a high level of supervision or externally mediated motivation that would cause an extensive caloric deficit, resulting in behavioral changes so great as to otherwise obviate the appetite-reducing or food intake-reducing activity of the composition administered. It is further contemplated by the inventors that the weight loss program of the present invention should be of sufficient intensity to result in demonstrable weight loss over a reasonable length of program, such as about three months. This provides motivation for the treated individual to continue the weight loss program and continue consumption of the comestible fiber product that is an adjuvant to the weight loss effort. Although the amount of weight loss needed to maintain sufficient motivation could obviously vary greatly among individuals, about one pound per month will be appropriate for many individuals. Again, if this caloric deficit were achieved solely be decreased food intake, a decrease in caloric intake of about 120 kcal per day is needed to achieve this rate of weight loss. This decreased food intake may also be achieved by a combination of decreased food intake and increased exercise, or solely by an increase in exercise.

Although shown and described is what is believed to be the most practical and preferred embodiments, it is apparent that departures from specific designs and methods described and shown will suggest themselves to those skilled in the art and may be used without departing from the spirit and scope of the invention. The present invention is not restricted to the particular constructions described and illustrated, but should be constructed to cohere with all modifications that may fall within the scope of the appended claims.

What is claimed is:

1. A method for reducing weight in an individual comprising adhering to a weight loss program that provides a caloric deficit of not more than about 350 kilocalories per day, and concurrently ingesting a composition comprising effective amounts of a combination of an anionic soluble fiber and a multivalent cation source during the weight loss program.

2. A method for reducing weight of claim 1 wherein the caloric deficit is achieved by reducing food intake, in comparison to the food intake prior to beginning the weight loss program.

3. A method for reducing weight of claim 2, wherein the caloric deficit is from about 120 to about 350 kilocalories per day.

4. A method for reducing weight of claim 3, wherein the caloric deficit is from about 150 to about 300 kilocalories per day.

5. A method for reducing weight of claim 4, wherein the caloric deficit if from about 160 to about 270 kilocalories per day.

6. A method for reducing weight of claim 1, wherein the individual has previously completed a weight loss program comprising an exercise component and a counseling component.

7. A method for reducing weight of claim 6, wherein the previous weight loss program comprising an exercise component and a counseling component was completed within 30 days of starting the weight loss program.

8. A method for reducing weight of claim 1 wherein the comestible product contains an anionic soluble fiber and a multivalent cation source.

9. A method for reducing weight of claim 8 wherein the anionic soluble fiber is alginate.

10. A method for reducing weight of claim 8 wherein the multivalent cation source is selected from the group consisting of calcium, magnesium, aluminum, manganese, iron, nickel, copper, zinc, strontium, barium, bismuth, chromium, vanadium, and lanthanum, their salts, and mixtures thereof.

11. A method of reducing weight of claim 10 wherein the multivalent cation salt is selected from the group consisting of formate, fumarate, acetate, propionate, butyrate, caprylate, valerate, lactate, citrate, malate and gluconate, chloride, phosphate and mixtures thereof.

12. A method for reducing weight of claim 10, wherein the multivalent cation is calcium and wherein the salt is selected from the group consisting of calcium citrate, calcium tartrate, calcium succinate, calcium fumarate, calcium adipate, calcium malate, calcium lactate, calcium gluconate, dicalcium phosphate dihydrate, anhydrous calcium diphosphate, dicalcium phosphate anhydrous, calcium chloride, calcium acetate monohydrate, and mixtures thereof.

13. A method for reducing weight of claim 8, wherein a ratio of the soluble anionic fiber to the multivalent cation in the comestible product is from about 20:1 to 7:1.

14. A method of claim 1 for reducing weight, wherein the amount of soluble anionic fiber is from about 1.5 grams to about 6 grams.

15. A method for reducing weight of claim 1, wherein the ingestible composition contains from about 50 kilocalories to 150 kilocalories.

16. A method for reducing weight of claim 1, wherein the ingestible composition is selected from a formed solid, a fluid, and a combination thereof.

17. A method for reducing weight loss of claim 1, wherein the weight loss program comprises low intensity counseling.

18. A method of claim 1 wherein the composition comprising effective amounts of a combination of an anionic soluble fiber and a multivalent cation source is consumed about twice per day during the weight loss program.

19. A method for producing weight loss in an individual, the method comprising identifying an individual desiring or in need of weight loss, providing to the individual a weight loss program that provides a caloric deficit of not more than about 300 kilocalories per day, and directing the individual during the weight loss program to ingest a composition comprising an effective amount of a combination of an anionic soluble fiber and a multivalent cation.

20. A method for producing weight loss of claim 19 wherein the caloric deficit is achieved by reducing food intake, in comparison to the food intake prior to beginning the weight loss program.

21. A method for producing weight loss of claim 19, wherein the caloric deficit is from about 120 to about 350 kilocalories per day.

22. A method for producing weight loss of claim 21, wherein the caloric deficit is from about 150 to about 300 kilocalories per day.

23. A method for producing weight loss of claim 22, wherein the caloric deficit if from about 160 to about 270 kilocalories per day.

24. A method for producing weight loss of claim 19, wherein the individual has previously completed a weight loss program comprising an exercise component and a counseling component.

25. A method for producing weight loss of claim 19, wherein the weight loss program comprises low intensity counseling.
